(19) United States
(12) Patent Application Publication
(10) Pub. No.: US 2021/0169568 A1
(43) Pub. Date: Jun. 10, 2021

(54) ORIENTED IRREVERSIBLE-ELECTROPORATION (IRE) PULSES TO COMPENSATE FOR CELL SIZE AND ORIENTATION

(71) Applicant: BIOSENSE WEBSTER (ISRAEL) LTD., Yokneam (IL)

(72) Inventors: ASSAF GOVARI, Haifa (IL); ANDRES CLAUDIO ALTMANN, Haifa (IL)

(21) Appl. No.: 16/707,371

(22) Filed: Dec. 9, 2019

(51) Int. Cl. A61B 18/14; A61B 18/12; A61B 18/18; A61B 18/00

(52) U.S. Cl. CPC A61B 18/1492; A61B 18/1206; A61B 2018/00613; A61B 2018/0022; A61B 2018/1861

(57) ABSTRACT

A system includes an irreversible electroporation (IRE) pulse generator, a switching assembly, and a processor. The IRE pulse generator is configured to generate IRE pluses. The switching assembly is configured to deliver the IRE pulses to multiple electrodes that are disposed on an expandable distal end of a catheter that is placed in contact with tissue in an organ, for applying the IRE pulses to the tissue. The processor is configured to (a) receive one or more prespecified orientations along which electric fields in the tissue are to be generated by the IRE pulses, (b) select one or more pairs of the electrodes that would apply the IRE pulses at the prespecified orientations, and (c) connect the IRE pulse generator, using the switching assembly, to the selected pairs of the electrodes.

### FIELD OF THE INVENTION

The present invention relates generally to invasive medical probes, and particularly to balloon catheters for irreversible electroporation.

### BACKGROUND OF THE INVENTION

Delivery of irreversible electroporation (IRE) energy to tissue using multi-electrode catheters was previously proposed in the patent literature. For example, U.S. Pat. No. 9,289,606 describes catheter systems that include direction-sensitive, multi-polar tip electrode assemblies for electroporation-mediated therapy, electroporation-induced primary necrosis therapy and electric field-induced apoptosis therapy, including configurations for producing narrow, linear lesions as well as distributed, wide area lesions.

As another example, U.S. Patent Application Publication 2019/0030328 describes a medical device configured to electroporate an area of tissue, the medical device including a balloon having a distal portion and a proximal portion, and a plurality of electrodes disposed on the distal portion of the balloon, each of the plurality of electrodes being configured to deliver electroporation energy to the area of tissue.

U.S. Pat. No. 8,992,517 describes methods, devices, and systems for in vivo treatment of cell proliferative disorders. The invention can be used to treat solid tumors, such as brain tumors. The methods rely on non-thermal irreversible electroporation (IRE) to cause cell death in treated tumors. The method encompasses the use of multiple electrodes and different voltages applied for each electrode to precisely control the three-dimensional shape of the electric field for tissue ablation. More specifically, it has been found that varying the amount of electrical energy emitted by different electrodes placed in a tissue to be treated allows the practitioner to finely tune the three-dimensional shape of the electrical field that irreversibly disrupts cell membranes, causing cell death. Likewise, the polarity of electrodes can be varied to achieve different three-dimensional electrical fields.

### SUMMARY OF THE INVENTION

An exemplary embodiment of the present invention provides a system including an irreversible electroporation (IRE) pulse generator, a switching assembly, and a processor. The IRE pulse generator is configured to generate IRE pluses. The switching assembly is configured to deliver the IRE pulses to multiple electrodes that are disposed on an expandable distal end of a catheter that is placed in contact with tissue in an organ, for applying the IRE pulses to the tissue. The processor is configured to (a) receive one or more prespecified orientations along which electric fields in the tissue are to be generated by the IRE pulses, (b) select one or more pairs of the electrodes that would apply the IRE pulses at the prespecified orientations, and (c) connect the IRE pulse generator, using the switching assembly, to the selected pairs of the electrodes.

In some exemplary embodiments, each of the electrodes includes a plurality of electrode segments, and wherein the switching assembly and the processor are configured to individually include any of the electrode segments in the one or more pairs.

In some exemplary embodiments, the electrodes are disposed equiangularly about a longitudinal axis of the distal end.

In an exemplary embodiment, the processor is configured to select first and second pairs of the electrodes along mutually orthogonal orientations. In another exemplary embodiment, the one or more prespecified orientations are prespecified relative to a longitudinal axis of the distal end.

In some exemplary embodiments, the processor is configured to apply the IRE pulses by applying bi-phasic IRE pulses.

There is additionally provided, in accordance with an exemplary embodiment of the present invention, a method including placing multiple electrodes of an expandable distal end of a catheter in contact with a tissue in an organ for applying IRE pulses to tissue. Irreversible electroporation (IRE) pluses are generated using an IRE pulse generator. One or more prespecified orientations are received along which electric fields in tissue are to be generated by the IRE pulses. One or more pairs of the electrodes are selected that would apply the IRE pulses at the prespecified orientations. The IRE pulses are applied to the tissue at the prespecified orientations, by connecting the IRE pulse generator to the selected pairs of the electrodes.

There is further provided, in accordance with an exemplary embodiment of the present invention, a system including an irreversible electroporation (IRE) pulse generator, a switching assembly, and a processor. The IRE pulse generator is configured to generate IRE pluses. The switching assembly is configured to deliver the IRE pulses to multiple electrodes that are disposed on an expandable distal end of a catheter that is placed in contact with tissue in an organ, for applying the IRE pulses to the tissue. The processor is configured to select first and second pairs of the electrodes that would apply the IRE pulses to a same region of tissue at two orientations which are not parallel to each other, and connect the IRE pulse generator, using the switching assembly, to the selected first and second pairs of the electrodes.

### BRIEF DESCRIPTION OF THE DRAWINGS

The present invention will be more fully understood from the following detailed description of the embodiments thereof, taken together with the drawings in which:

FIG. 1 is a schematic, pictorial illustration of a catheter-based irreversible electroporation (IRE) system, in accordance with an exemplary embodiment of the present invention;

FIG. 2 is a schematic, pictorial side view of the irreversible electroporation (IRE) balloon catheter of FIG. 1 deployed in a region of a pulmonary vein (PV) and its ostium, in accordance with an exemplary embodiment of the invention; and

FIG. 3 is a flow chart that schematically illustrates a method for applying directional IRE pulses using the IRE balloon catheter of FIG. 2, in accordance with an exemplary embodiment of the present invention.

### DETAILED DESCRIPTION OF EMBODIMENTS

### Overview

Irreversible electroporation (IRE), also called Pulsed Field Ablation (PFA), may be used as an invasive therapeutic modality to kill tissue cells by subjecting them to high-voltage pulses. IRE may be associated with DC pulses or mono-phasic pulses, where when IRE ablation is referred to as PFA (Pulsed Field Ablation), bi-phasic IRE pulses are used. However, the term IRE may be used to refer to any type of the above-mentioned pulse shapes.

Specifically, IRE pulses have a potential use to kill myocardium tissue cells in order to treat cardiac arrhythmia. Of particular interest is the use of bipolar electric pulses (e.g., using a pair of electrodes in contact with tissue) to kill tissue cells between the electrodes. Cellular destruction occurs when the transmembrane potential exceeds a threshold, leading to cell death and thus the development of a tissue lesion.

Myocardium tissue includes specialized myocardial cells that conduct electrophysiological signals. For example, a collection of these specialized myocardial cells, the sinus node, initiates the heartbeat. Each myocardial cell is typically long and thin. Cardiac tissue comprises multiple myocardial cells that are aggregated into so-called myofibers of conduction tissue. The alignment in space of the myofibers of conduction tissue (i.e., the orientation of the myocardial cells) largely depends on their location in the heart.

Cell death is caused by the applied electrical field, and different cells react differently to different field levels, i.e., have different thresholds for being killed. In addition, the way a non-spherical cell responds to the applied electrical field depends on the geometrical orientation of the cell with respect to the field. Myocardial cells have relatively large ellipsoidal eccentricity, being about 100 μm long and 10-25 μm in diameter. Therefore, while IRE may be used to kill myocardial cells, the non-spherical cell shape of cells means that knowledge of the cell orientation is needed for setting an optimal lethal electric field.

Exemplary embodiments of the present invention that are described hereinafter use a catheter with multiple electrodes which may be selected to generate different electric fields (in magnitude and direction). To overcome not knowing the myocardial cell orientation in the vicinity of the electrodes, in some exemplary embodiments the electric field is applied in at least two different orientations, typically orthogonal to each other. This reduces the pulse voltage amplitude needed, since otherwise, a high pulse voltage is needed to overcome a “worst case” scenario of a field-cell alignment along the elongated direction of cells. If the myocardial cell orientation is known (typically, by other means) the configuration of the electrodes used to generate the killing electrical field may be optimized.

In some exemplary embodiments, a medical probe having an expandable frame disposed with a plurality of electrodes, such as balloon catheter or a basket catheter, is used to apply the high voltage pulses along two approximately orthogonal orientations in multiple locations over the expandable frame, as described below. To enable the application of the directional electric fields, the plurality of electrodes is connected to an output of an IRE pulse generator via a processor-controlled switching box (also referred to as switching assembly).

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20 percent of the recited value, e.g. “about 90 percent” may refer to the range of values from 71 percent to 99 percent.

In an embodiment, before application of bipolar IRE pulses by multiple pairs of electrodes, the processor receives one or more prespecified orientations (e.g., relative to the longitudinal axis of the distal end) along which electric fields in tissue should be generated by the IRE pulses. The processor accordingly determines a configuration of electrode pairs over the expandable frame. Then the processor controls the switching box to connect the electrodes according to the determined configuration, i.e., to connect the electrodes to the IRE pulse generator to apply the IRE pulses between the electrodes along the one or more prespecified orientations.

For example, if the myofibers of a tissue of a lumen of a vessel is known to be aligned longitudinally (i.e., along the lumen) over an entire perimeter of the wall tissue of the lumen, then the electrode pairs are configured to generate a locally transverse electric field between each electrode pair.

By applying IRE pulses of an electrical field along orthogonal orientations or along a prespecified direction, the disclosed catheter-based IRE treatment techniques increase tissue selectivity to treatment, and thus may improve the clinical outcome of invasive IRE treatments, such as of an IRE treatment of cardiac arrhythmia.

### System Description

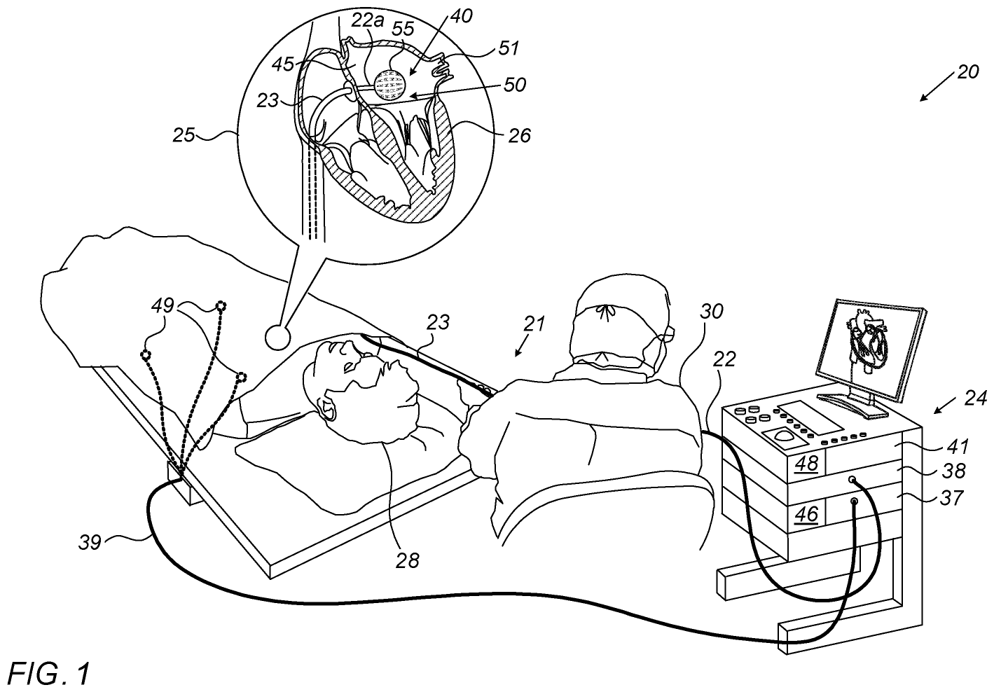

FIG. 1 is a schematic, pictorial illustration of a catheter-based irreversible electroporation (IRE) system 20, in accordance with an exemplary embodiment of the present invention. System 20 comprises a catheter 21, wherein a shaft 22 of the catheter is inserted into a heart 26 of a patient 28 through a sheath 23. The proximal end of catheter 21 is connected to a console 24.

Console 24 comprises an IRE generator 38 configured to generate IRE pulses. The IRE pulses are delivered via catheter 21 to ablate tissue in a left atrium 45 of heart 26. For example, the IRE pulses may be bi-phasic pulses shaped as a positive pulse section (e.g., of +1000V) followed by a negative pulse section (e.g., of −1000V).

In the exemplary embodiment described herein, catheter may be used for any suitable therapeutic and/or diagnostic purpose, such as electrical sensing and/or IRE isolation of ostium 51 tissue of a pulmonary vein in left atrium 45 of heart 26.

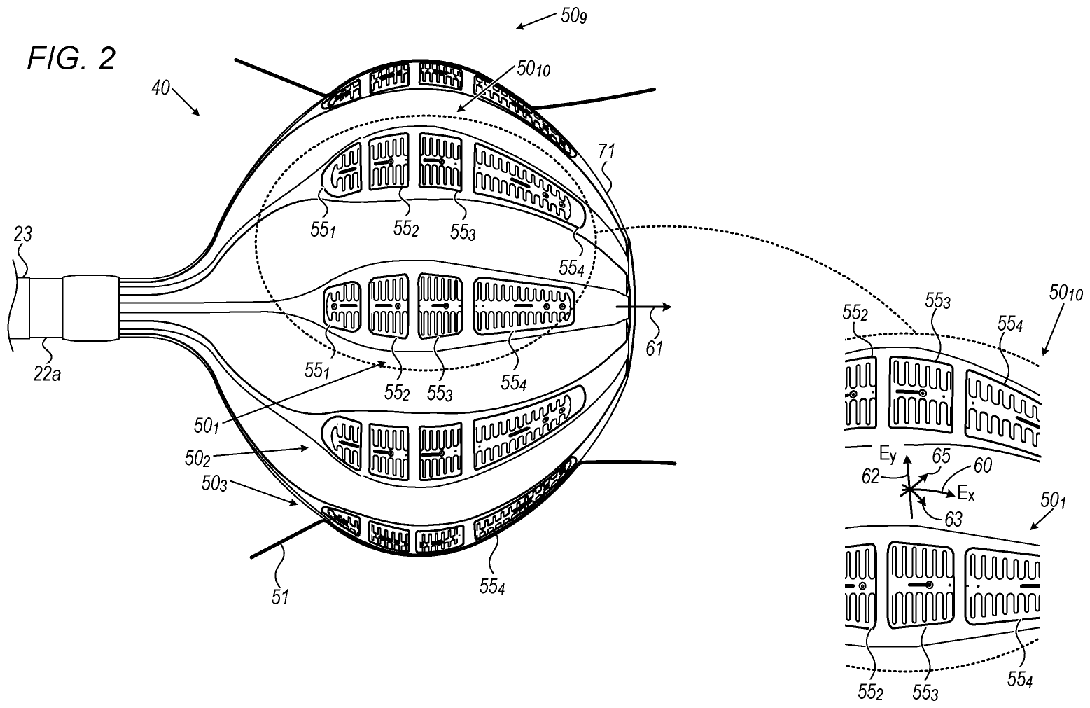

A physician 30 inserts shaft 22 through the vascular system of patient 28. As seen in inset 25, an expandable balloon catheter 40, fitted at a distal end 22a of shaft 22, comprises multiple IRE electrodes 50, further described in FIG. 2. During the insertion of shaft 22, balloon 40 is maintained in a collapsed configuration inside sheath 23. By containing balloon 40 in a collapsed configuration, sheath 23 also serves to minimize vascular trauma along the way to target location. Physician 30 navigates the distal end of shaft 22 to a target location in the heart 26.

Once distal end 22a of shaft 22 has reached the target location, physician 30 retracts sheath 23 and expands balloon 40, typically by pumping saline into the balloon 40. Physician 30 then manipulates shaft 22 such that electrodes 55 disposed on balloon catheter 40 engage an interior wall of the ostium to apply directional high-voltage IRE pulses via electrodes 50 to ostium 51 tissue. To apply directional IRE pulses, electrodes 50 are divided into segments 55, so as to form a largely two-dimensional array of electrode segments about each location over balloon 40, as further described in FIG. 2.

Console 24 includes a switching box 46 (also referred to as a switching assembly) that can switch any segment 55 of a segmented electrode 50 between acting as part of a pair of electrode segments that applies an electric field in a given direction or in an approximately orthogonal direction to the given direction, as described below.

Electrodes 50 are connected by wires running through shaft 22 to processor 41 controlling switching box 46 of interface circuits 37 in the console 24. Directional IRE protocols comprising IRE parameters such as electrode segment pair configurations are stored in a memory 48 of the console 24.

Console 24 comprises a processor 41, typically a general-purpose computer, with suitable front end and interface circuits 37 for receiving signals from catheter 21 and from external electrodes 49, which are typically placed around the chest of patient 28. For this purpose, processor 41 is connected to external electrodes 49 by wires running through a cable 39.

Processor 41 is typically programmed (software) to carry out the functions described herein. The software may be downloaded to the computer in electronic form, over a network, for example, or it may, alternatively or additionally, be provided and/or stored on non-transitory tangible media, such as magnetic, optical, or electronic memory.

Although the illustrated exemplary embodiment relates specifically to the use of a balloon for IRE of heart tissue, the elements of system 20 and the methods described herein may alternatively be applied to control ablation using other sorts of multi-electrode ablation devices, such as a basket catheter that carries multiple electrodes on spines of an expandable frame.

### Oriented IRE Pulses to Compensate for Cell Size and Orientation

FIG. 2 is a schematic, pictorial side view of the irreversible electroporation (IRE) balloon catheter 40 of FIG. 1 deployed in a region of a pulmonary vein (PV) and its ostium 51, in accordance with an exemplary embodiment of the present invention. The balloon catheter 40 is used to ablate ostium 51 tissue to isolate a source of arrhythmia. Balloon 40 has ten segmented electrodes 50 (50₁ . . . 50₁₀) disposed over a membrane 71 of the balloon.

Bipolar IRE pulses can be delivered from IRE generator 38 independently to each pair of segments 55 (55₁ . . . 55₄) of each of the ten electrodes 50, either between segments of the same electrode or between segments of neighboring electrodes. When the bipolar IRE pulse is applied between segments of a same electrode 50, it creates an electric field approximately parallel with a longitudinal axis 61, defined by distal end 22a of shaft 22. For example, a bipolar pulse applied between segments 55₂ and 55₃ of electrode 50₁₀ and a bipolar pulse applied between segments 55₂ and 55₃ of electrode 50₁, generate electrical fields E. 60 at different tissue locations in contact with balloon 40 over a perimeter of balloon 40. Both these fields are parallel to longitudinal axis 61.

When the bipolar IRE pulse is applied between corresponding segments 55 of neighboring electrodes 50 it creates an electric field approximately parallel with an azimuthal axis, or a locally transverse axis, y. For example, a bipolar pulse applied between segment 55₂ of electrode 50₁₀ and segment 55₂ of electrode 50₁, and a bipolar pulse applied between segment 55₃ of electrode 50₁₀ and segment 55₃ of electrode 50₁, generate electrical fields E_{y} 62 at different tissue locations in contact with balloon 40 over a perimeter of balloon 40. Both these fields are orthogonal to longitudinal axis 61.

In some exemplary embodiments the segments are connected, using switching box 46, to create orthogonal fields that are tilted relative to longitudinal axis 61. For example, a bipolar pulse applied between segment 55₂ of electrode 50₁₀ and segment 55₄ of electrode 50₁ creates an electric field 63 that is approximately orthogonal to an electric field 65 created by a bipolar pulse applied between segment 55₂ of electrode 50₁ and segment 55₄ of electrode 50₁₀, with the two fields rotated approximately (+45) degrees and (−45) degrees, respectively, relative to longitudinal axis 61.

In the exemplary embodiment shown in FIG. 2, the balloon catheter comprises forty segments 55 (four per electrode), although the number and shape of the segments may differ.

Processor 41 controls switching box 46 to connect the segment pairs according, for example, to a prespecified configuration applied in an IRE balloon treatment protocol of a given cardiac tissue.

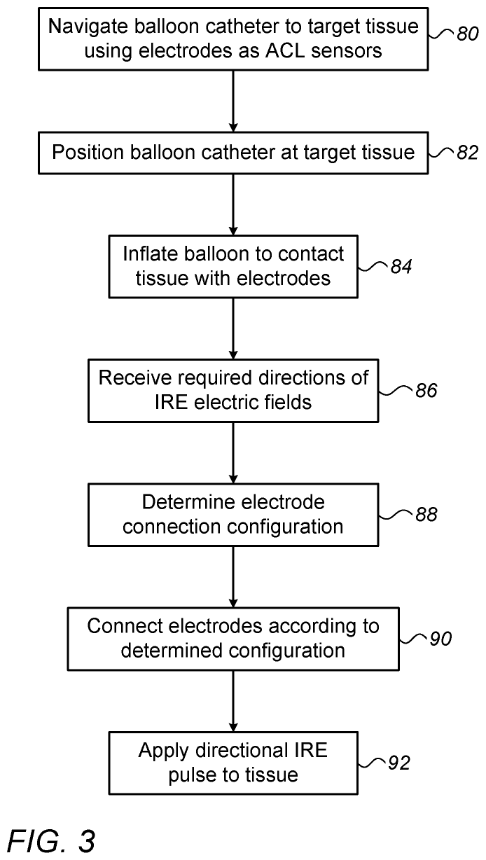

FIG. 3 is a flow chart that schematically illustrates a method for applying directional IRE pulses using the balloon of FIG. 2, in accordance with an exemplary embodiment of the present invention. The algorithm, according to the presented exemplary embodiment, carries out a process that begins when physician 30 navigates the balloon catheter to a target tissue location in an organ of a patient, such as at ostium 51, using, for example, electrode 50 as ACL sensing electrodes, at a balloon catheter navigation step 80.

Next, physician 30 positions the balloon catheter at ostium 51, at a balloon catheter positioning step 82. Next, physician 30 fully inflates balloon 40 to contact target tissue with electrodes 50 over an entire circumference of the lumen, at a balloon inflation step 84.

Next, at an IRE planning step 86, processor 41 receives one or more prespecified orientations (e.g., relative to the longitudinal axis of the distal end) along which the IRE pulses should generate an electric field in the tissue. For example, initial orientations are received from a protocol and are adjusted by the position tracking system before being received in the processor. The prespecified orientations may differ from one region to another around ostium 51.

Based on the required orientations, processor 41 determines an electrode connection configuration, examples of which are described in FIG. 2, at an electrode configuration setup step 88.

Next, processor 41 controls switching box 46 to connect the electrodes according to the determined configuration, at an electrode connecting step 90.

Finally, processor 41 applies the directional IRE pulses to tissue, at an IRE treatment step 92.

The flow chart of FIG. 3 is an exemplary flow that is depicted purely for the sake of clarity. In alternative embodiments, any other suitable method flow may be used. For example, the method of FIG. 2 assumes that the orientations of the myocardial cells are known, i.e., that there is sufficient information for specifying the IRE pulse orientations at step 86. In alternative exemplary embodiments, e.g., in the absence of sufficient information regarding myocardial cell orientations, processor 41 may control switching box 46 to apply IRE pulses at multiple (typically two) different orientation to the same region of tissue. For example, processor 41 may control switching box 46 to apply IRE pulses having orthogonal orientations, e.g., one bipolar pulse between segment 55₂ of electrode 50₁₀ and segment 55₄ of electrode 50₁, and another bipolar pulse between segment 55₂ of electrode 50₁ and segment 55₄ of electrode 50₁₀. Any other suitable configuration can also be applied.

Although the exemplary embodiments described herein mainly address cardiac applications, the methods and systems described herein can also be used in other medical applications, such as to treat different type of cancers, for example lung cancer and liver cancer, and in neurology and otolaryngology.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for irreversible electroporation, the system comprising:
an irreversible electroporation (IRE) pulse generator configured to generate IRE pluses;
a switching assembly, configured to deliver the IRE pulses to multiple electrodes that are disposed on an expandable distal end of a catheter that is placed in contact with tissue in an organ, for applying the IRE pulses to the tissue; and
a processor, which is configured to:
receive one or more prespecified orientations along which electric fields in the tissue are to be generated by the IRE pulses;
select one or more pairs of the electrodes that would apply the IRE pulses at the prespecified orientations; and
connect the IRE pulse generator, using the switching assembly, to the selected pairs of the electrodes.

2. The system according to claim 1, wherein each of the electrodes comprises a plurality of electrode segments, and wherein the switching assembly and the processor are configured to individually include any of the electrode segments in the one or more pairs.

3. The system according to claim 1, wherein the electrodes are disposed equiangularly about a longitudinal axis of the distal end.

4. The system according to claim 1, wherein the processor is configured to select first and second pairs of the electrodes along mutually orthogonal orientations.

5. The system according to claim 1, wherein the one or more prespecified orientations are prespecified relative to a longitudinal axis of the distal end.

6. The system according to claim 1, wherein the processor is configured to apply the IRE pulses by applying bi-phasic IRE pulses.

7. A method for irreversible electroporation, comprising:
placing multiple electrodes of an expandable distal end of a catheter in contact with a tissue in an organ for applying IRE pulses to tissue;
generating irreversible electroporation (IRE) pluses using an IRE pulse generator;
receiving one or more prespecified orientations along which electric fields in tissue are to be generated by the IRE pulses; and
selecting one or more pairs of the electrodes that would apply the IRE pulses at the prespecified orientations; and
applying the IRE pulses to the tissue at the prespecified orientations, by connecting the IRE pulse generator to the selected pairs of the electrodes.

8. The method according to claim 7, wherein each of the electrodes comprises a plurality of electrode segments, and wherein selecting the pairs comprises individually including any of the electrode segments in the one or more pairs.

9. The method according to claim 7, wherein the electrodes are disposed equiangularly about a longitudinal axis of the distal end.

10. The method according to claim 7, wherein selecting the pairs comprises selecting first and second pairs of the electrodes along mutually orthogonal orientations.

11. The method according to claim 7, wherein applying apply the IRE pulses comprises applying bi-phasic IRE pulses.

12. A system for irreversible electroporation, comprising:
an irreversible electroporation (IRE) pulse generator configured to generate IRE pluses;
a switching assembly, configured to deliver the IRE pulses to multiple electrodes that are disposed on an expandable distal end of a catheter that is placed in contact with tissue in an organ, for applying the IRE pulses to the tissue; and
a processor, which is configured to:
select first and second pairs of the electrodes that would apply the IRE pulses to a same region of tissue at two orientations which are not parallel to each other; and
connect the IRE pulse generator, using the switching assembly, to the selected first and second pairs of the electrodes.

13. The system according to claim 12, wherein the processor is configured to select the first and second pairs of the electrodes along approximately mutually orthogonal orientations.
